(19) United States
(12) Patent Application Publication
(10) Pub. No.: US 2019/0201640 A1
(43) Pub. Date: Jul. 4, 2019

(54) DEVICE FOR DISPENSING AN AIR-DISCHARGEABLE SUBSTANCE

(71) Applicant: Alfred VON SCHUCKMANN, Kevelaer (DE)

(72) Inventor: Alfred VON SCHUCKMANN, Kevelaer (DE)

(21) Appl. No.: 16/314,076

(22) PCT Filed: Jul. 3, 2017
(86) PCT No.: PCT/EP2017/066436

§ 371 Date: Dec. 28, 2018

(30) Foreign Application Priority Data
Jul. 4, 2016 (DE) 10 2016 008 015.9
Oct. 18, 2016 (DE) 10 2016 119 789.0

(51) Int. Cl. A61M 15/00

(52) U.S. Cl. CPC A61M 15/0041; A61M 2206/20; A61M 2202/064

(57) ABSTRACT

A device for dispensing an air-dischargeable substance includes an air inlet, a substance-loaded air discharge nozzle, an outflow region having a chamber containing the substance, a first air guide duct from the outflow region to the nozzle, and a second air guide duct from the inlet to the outflow region fluidically connected, via a connection channel, to the first air guide duct. Air flows in through an inflow opening through the connection channel into the first air guide duct perpendicularly to the substance-loaded air flow direction into the first air guide duct in the inflow-opening region. The outflow region has a partition of hard material between the first and second air guide ducts with a soft material free end face facing the chamber. The chamber includes a foil cover overlapping with a chamber cavity containing the substance against which the partition lies forming a seal via the soft material.

### CROSS REFERENCE TO RELATED APPLICATIONS

This application is the National Stage of PCT/EP2017/066436 filed on Jul. 3, 2017, which claims priority under 35 U.S.C. § 119 of German Application No. 10 2016 008 015.9 filed on Jul. 4, 2016 and German Application No. 10 2016 119 789.0 filed on Oct. 18, 2016, the disclosures of which are incorporated by reference. The international application under PCT article 21(2) was not published in English.

### FIELD OF THE INVENTION

The invention relates to a device for dispensing an air-dischargeable substance, wherein reference is made to an inhaler in a special regard, for discharge of a pharmaceutical substance.

Such a device has an air inlet and a discharge nozzle. The discharge nozzle may be designed as a mouthpiece for application of suction by a human user. Substance-loaded air can be discharged through the discharge nozzle, in particular as a result of applying suction air.

The device also preferably serves to dispense substance present in a chamber. The chamber may be designed in the form of a pot, preferably made of a hard plastic, optionally also formulated to be transparent. The chamber may have a film-type cover that can preferably be punctured for opening. The chamber may also be a blister chamber or part of a blister strip. It may also be a device for a single emptying, after to which the device is to be opened and a new chamber is to be inserted, or it may be a device with which several chambers can be introduced into the outflow region successively, preferably incrementally, for example, by means of a blister strip.

### STATE OF THE ART

With regard to the state of the art, reference is made first to WO 2010/084415 A1 (US Patent 2012/0037158 A1), additionally to WO 01/26720 A1 (U.S. Pat. No. 6,880,555 B1).

GB 2407042 A discloses a device for output of an air-dischargeable substance, in which an inflow opening having a circular edge contour is formed. A comparable state of the art is also described in EP 2082764 A1.

WO2014/006135 A2 discloses a device, in which the first air guide duct is combined with ribs of the pivotable device part extending as a flat surface in the closed state of the device, utilizing a top side of the chamber to be emptied. A comparable state of the art is known from DE 10 2008 023 376 A1. In addition, reference is also made to DE 10 2014 005 646 A1, DE 10 2009 041 664 A1 and WO 2015/110832 A1.

### SUMMARY OF THE INVENTION

A first target direction of the invention relates to advantageous emptying of the chamber. Another target direction of the invention also relates to an advantageous design of the device as such. Another target direction also relates to the finest possible dispersion of substance in the air to be discharged. This is also the case with a simple design of the device in particular.

The device preferably additionally has an outflow region, in which a chamber with substance to be discharged is or may be arranged. A first air guide duct may lead from the outflow region to the discharge nozzle. In addition, a second air guide duct may lead from the air inlet to the outflow region.

The second air guide duct may be fluidically connected to the first air guide duct via a connecting duct. The connecting duct may form a bypass, through which air can flow, preferably free of substance. The air flow, which can be loaded with substance, may be guided via an additional flow path formed essentially by the two connecting ducts.

An inflow direction of air flowing through an inflow opening in the first air guide duct may be directed perpendicular to a direction of flow of the substance-loaded air in the first air guide duct in the region of the inflow opening. Due to air flows encountering one another, in particular in the region of the inflow opening, there is advantageous turbulence in the region of the discharge nozzle. This achieves a further disintegration of the substance, which can produce the very small particles desired as being favorable for inhalation.

It is also possible for just one (quantifier) such connecting channel to be provided.

The inflow opening, which is provided at the bottom, may also be designed as a slot-shaped opening provided across the direction of flow of the substance-loaded air, extending over a significant portion of the width, preferably more than 50% of the width, considered across the direction of flow, up to 90% or 100% of the width of the first air guide duct. In particular the wall (ceiling) of the first air guide duct is preferably designed to be closed opposite the inflow opening.

In the outflow region, a partition may be formed between the first and second air guide ducts. The partition may be made of a hard material, in particular a hard plastic. The partition may be part of the device housing and may extend across the direction of flow.

A free end face of the partition facing the chamber may be provided with a soft material for an advantageous seal. The soft material may be rubber or a TPE (thermoplastic elastomer), for example. The end face may run in coverage to a film cover of the chamber, for example. The end face may traverse a chamber cavity in an installation situation, so that in an imaginary continuation of the end face into the chamber, this would result in a division of the chamber cavity, wherein substance is then situated in any such partial chamber. The end face may form a tight seal with a film cover of the chamber when in use.

The chamber receptacle of the device may be formed in a first housing part, and a second housing part, which is pivotably connected to the first housing part, may be provided.

According to another teaching of the invention, the air guide duct may be designed so that it is divided longitudinally over at least a portion of its length and as seen in its longitudinal direction. A first subregion and a second subregion of the air guide duct may be formed on the first and second housing parts. Thus, an air guide duct, having a completely closed cross section, may be provided over at least a portion of its length only when the second housing part is in a proper use position relative to the first housing part. An air guide duct leads from the emptying area to the discharge nozzle, and a second air guide duct leads from the air inlet to the emptying area. The first air guide duct and the second air guide duct are designed so they are divided over a portion of their length at any rate, wherein a first subregion of the second air guide duct and a second subregion of the first air guide duct are provided on the first housing part, and a third subregion of the second air guide duct and a fourth subregion of the first air guide duct are provided on the second housing part. The two subregions of the second air guide duct and the first air guide duct can be formed separately into recognizable area regions not belonging to an air guide duct as part of a planar surface that does not otherwise go beyond this. The additional subregions may be designed in chambers in which a cover or bottom is missing in the open condition, and the closed first and second air duct channels are formed only by the interaction in the closed condition. The third subregion and the fourth subregion can be supplemented to form the respective first and/or second air duct by pivoting the second housing part into a locked into a preferred locked position, but at any rate a position in contact with the first housing part. At the same time, a partition that is impermeable for the air flow can be moved into a contacting position relative to the chamber, with the chamber inserted, and a direct air flow from the second air guide duct can be suppressed by the partition in the first air guide duct, wherein the air flow runs only through the chamber in the use condition.

The chamber may have a puncturable chamber cover in the form of the aforementioned film cover, for example. A puncture device may be provided for forming at least one puncture opening in the chamber wall. With regard to the partition, which is preferably provided in the outflow region for deflection of air into the chamber, it is additionally possible to provide that the puncturing device has two puncture protrusions, one of which is designed to be associated with one side of the partition, and the other of which is designed to be associated with the other side of the partition. Thus, a puncture opening in the chamber cover may be provided on both sides, i.e., in front of and behind the partition with respect to the direction of flow. A puncture protrusion is adapted in cross section to an inside contour of a chamber wall presented as seen in the press-down direction. The chamber wall is formed in the shape of a ring in cross section, and the puncture protrusion has an outside contour in the shape of a segment of a circle. The puncture protrusion has a tip or cutting edge, which slides directly along an inside wall of the chamber in the wake of puncturing, in an area passing through the chamber cover in displacement of the puncture protrusion in a puncture direction.

The hard material mentioned in conjunction with the present patent application, in particular a hard plastic, may be different from a soft material, in particular a soft plastic, for example, with regard to its Shore-hardness, measured according to DIN 53505 of 2012, for example. In this regard, the hard plastic may have a Shore-hardness greater than 60, more preferably greater than 65 in particular, for example, a Shore-hardness in the range of 75 to 80.

Additional features of the invention are described below, also in the description of the figures, often in their preferred association with the concepts of the claims already explained above, but they may also be important in association with just one or more individual features, as described here, in particular the claims already discussed, or they may be important independently or in a completely different overall concept. It is also possible to provide for the measures of the claims already discussed to be combined.

For example, the partition may extend with its free end face in the installed position to the film cover of the chamber directed transversely to the direction of flow in the air guide duct. Thanks to the partition, a direct air flow from the second air guide duct into the first air guide duct can be suppressed. This is in particular also the result of possible sealing contact of the partition with a cover on the chamber side. The partition may be arranged here in such a way that it connects opposing wall regions of the air guide duct, as seen transversely to the direction of air flow. This connection preferably forms a complete seal. Since the partition itself is preferably also impermeable to air flow, the air flow may pass through the chamber only in the use condition.

The puncture device may be displaceable against a restoring force for puncture of the film cover of the chamber by the puncture protrusions. The restoring force may result from the material of the puncture device, for example, in the embodiment of the device made of a flexible material.

In another possible embodiment, the restoring force results from a spring. The design of the puncture device, made of a plastic material here, may involve an integral spring, for example, an integral spring arm. In addition, a thermoplastic elastomer material may apply the corresponding restoring force. In this regard, a conventional spring, for example, a steel spring, may also be provided, for example, in the form of a helical spring, additionally a cylindrical compression spring or a plate spring or the like, for example.

In another possible embodiment, the device may have a displaceable pushbutton for acting on the puncture device. In conventional use of the device, the pushbutton may be freely accessible from the outside, for example, for use with one or more fingers. The pushbutton may be held so that it is slidingly displaceable on the device, for example, being mounted on the second housing part.

In addition, the pushbutton may also be displaceable against the restoring force of a spring, for example, with entrainment of the puncture device in the direction of the chamber receptacle.

After puncturing the film cover of the chamber with the puncturing protrusions, the chamber may establish a flow connection from the first air guide duct to the second air guide duct. After the film cover has been punctured, the chamber is part of the entire air guide duct and forms the sole connection except for the short-circuit connection or bypass connection between the two partial air guide ducts.

The second housing part may be inseparably connected to the first housing part by a hinge. A geometric hinge axis may be aligned transversely to the direction of air flow. One possible deflection of the second air guide duct may be associated with the hinge area, and the hinge area may also preferably be provided in an end region of the device facing away from the discharge nozzle.

Downstream from the deflection in the direction of flow, the air flow may be in the opposite direction from the air flow upstream from the deflection. In addition, upstream from the deflection, the air may be guided only through the second air guide duct, and when considered in the direction of air flow downstream from the deflection, it may be guided through an additional subsection of the air guide duct and through the first air guide duct to the discharge nozzle.

The chamber receptacle may also be provided downstream from the deflection when considered in the direction of flow.

A puncture protrusion may be considered in a cross section, in particular a cross section across the direction of displacement of the protrusion, adapted to an internal contour of a chamber wall, which is presented as seen in the direction of pressing down. Thus, in one embodiment of the chamber wall, which is circular, as seen in such a cross section, the puncture protrusion may have an outside contour in the shape of a segment of a circle in the region passing through the chamber cover with displacement of the puncture protrusion in the puncture direction. This contour in the form of a segment of a circle may be based on the same midpoint as the circular contour with respect to the circle to be generated in the aforementioned cross section, in particular the inside contour of the chamber wall.

A puncture protrusion may also run in the form of a segment of a circle in the cross section on its side facing the central axis of the chamber. The puncture protrusion may have a thickness in the radial direction in this regard, corresponding to one-twentieth to one-fifth of the inside diameter of the chamber in a cross section in which the puncturing occurs.

An extent of a puncture protrusion in a circumferential direction with regard to the central axis of the chamber may extend approximately over one-tenth to one-third of the circumferential extent of a circle, which is formed in a cross section, in which the puncturing takes place and which coincides with the inside wall of the chamber.

A puncture protrusion may additionally form a tip, which may be shaped with a point, but also in the sense of a cutting edge running peripherally over at least a significant portion of the circumferential extent of the puncture protrusion. The tip or the cutting edge is also preferably designed, so that it runs directly along an inside wall of the chamber. In this embodiment, the puncture protrusion is enlarged accordingly from the tip or the cutting edge only toward the interior of the chamber. This enlargement may be designed in the form of a trailing sloping surface, such that the tip or the cutting edge forms the most forward region in the course of the puncturing.

Due to an embodiment with the tip or the cutting edge traveling along directly on the inside surface of the chamber, it is possible to achieve the result that the cover is separated from the chamber wall, starting from the inside with respect to the chamber. Thus, the cover on the chamber wall is at a distance, so that it is practically aligned with the chamber wall, and this is on both the inflow side and the outflow side. There may be air flow in particular directly along the chamber wall, over the bottom and again upward along the chamber wall, which can thus effectively support the desired emptying of the chamber as thoroughly as possible.

The cover of the chamber, for example, the film cover of the chamber, may also preferably be designed to be concave in a view as seen in the direction of puncture on the side of the cover on the outside. The resulting concave shape in the chamber cover may also fundamentally be inserted in the unloaded position of the cover. In another possible and even preferred embodiment, the concave shape is obtained when the chamber is inserted and optionally also only then, as a result of the application of force to the chamber cover by the partition. The end face of the partition may therefore be shaped so that it is adapted in the sealed area of exposure, forming a convex curvature in at least some sections accordingly.

### BRIEF DESCRIPTION OF THE DRAWINGS

The invention is described in greater detail below on the basis of the accompanying drawings, although they represent only embodiments. A part that is explained only with respect to one of the embodiments and in which additional embodiment it is not replaced by another part because of the particular features emphasized there (just) is thus also described for this additional embodiment as a part that is at any rate possibly present. In the drawings of the embodiment here:

FIG. 1 shows a side view of the unfolded device in a first embodiment, with a chamber to be inserted into the device;

FIG. 2 shows a view of the device according to FIG. 1, as seen in perspective without the chamber inserted;

FIG. 3 shows an enlarged diagram of the device according to FIG. 1 and/or FIG. 2 with the chamber inserted;

FIG. 4 shows a perspective longitudinal section through the device in the assembled condition and with the chamber inserted;

FIG. 5 shows a diagram according to FIG. 4 in a view from the outside;

FIG. 6 shows the device according to FIG. 4 and/or FIG. 5 in a view from the rear according to the arrow VI in FIG. 5;

FIG. 7 shows the device in a front view according to the arrow VII in FIG. 5;

FIG. 8 shows the device according to FIG. 4 and/or FIG. 5 in a view from the side;

FIG. 9 shows the device according to FIG. 4 in a perspective view obliquely from the rear;

FIG. 10 shows a cross section through the device according to FIG. 7, shown as a sectional view along the plane X-X;

FIG. 11 shows a diagram according to FIG. 4 with the puncture device depressed;

FIG. 12 shows a diagram according to FIG. 10 with the puncture device depressed;

FIG. 13 shows the device in a perspective diagram relating to a second embodiment;

FIG. 14 shows a perspective diagram relating to the device according to FIG. 13, corresponding essentially to FIG. 2;

FIG. 15 shows a perspective diagram relating to the device according to FIGS. 13 and 14, corresponding essentially to FIG. 3;

FIG. 16 shows the device of the second embodiment in a perspective exploded diagram;

FIG. 17 shows a longitudinal section through the device according to FIG. 13;

FIG. 18 shows the enlargement of the region XVIII from FIG. 17;

FIG. 19 shows a diagram corresponding to FIG. 18 with the puncture device depressed;

FIG. 20 shows the section along line XX-XX in FIG. 19;

FIG. 21 shows the section according to line XXI-XXI in

FIG. 20;

FIG. 22 shows a follow-up diagram of FIG. 19 relating to the device following an inhalation process.

### DESCRIPTION OF THE EMBODIMENTS

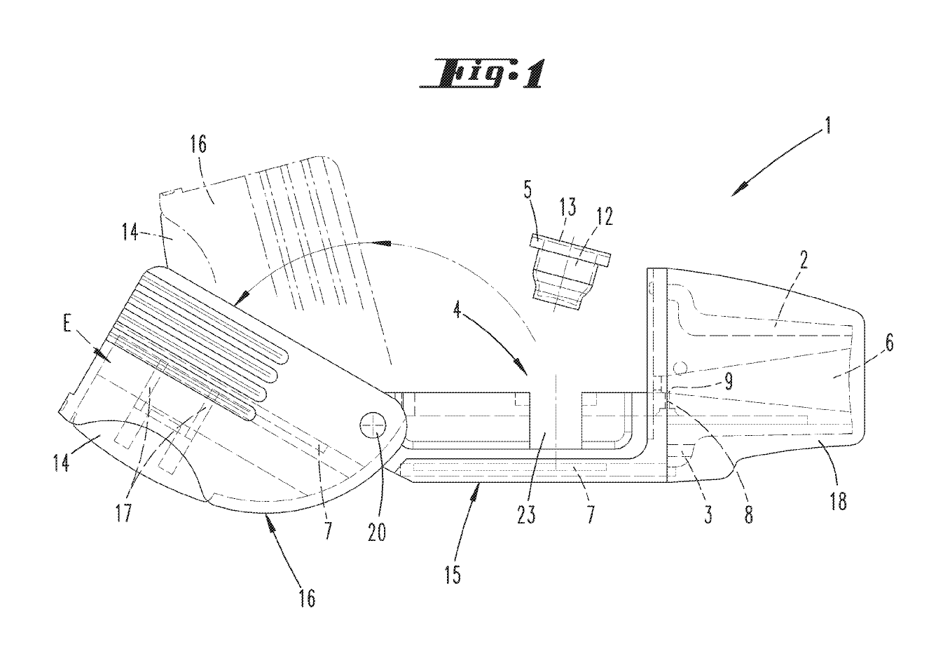

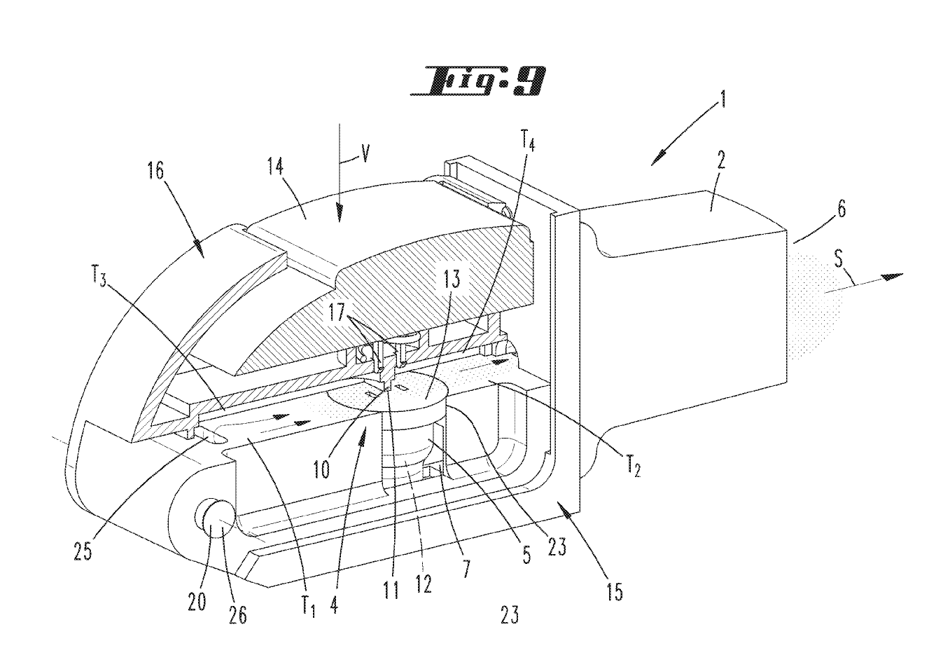

This illustrates and describes a device 1 (see FIGS. 1, 4 and 7) having a discharge nozzle 2 designed as a mouthpiece, an air inlet 3, which is preferably formed by a plurality of boreholes arranged side by side, and an outflow region 4, into which a chamber 5 filled with substance can be inserted. The substance, which is preferably a pharmaceutical substance, can be discharged by means of air, for example, by means of an air flow created by the user by applying suction.

The discharge nozzle 2 is covered by means of a cap 18 in a nonuse position of the device 1.

A first air guide duct 6 leads from the outflow region 4 to the discharge nozzle 2. A second air guide duct 7 leads from the air inlet 3 to the outflow region 4.

A connecting duct 8, by means of which the second air guide duct 7 is fluidically connected to the first air guide duct 6, is preferably formed between the second air guide duct 7 and the first air guide duct 6.

The connecting duct 8 is arranged at least approximately directly downstream from the air inlet 3, as seen in the direction of flow S. accordingly, the connecting duct 8 may also be arranged in the transitional region from the discharge nozzle 2 to the remaining device housing in the root area of the discharge nozzle 2.

The connecting duct 8 forms a bypass connection or a short-circuit connection next to the air guide duct discharging the substance.

Air flowing through the connecting duct 8 with an inflow direction R into the first air guide duct 6 is directed essentially at a right angle to the direction of flow S of the substance-loaded air, flowing in the first air guide duct 6 in the condition of use.

At any rate, this direction of flow S exists in the region of inflow opening 9 of the connecting duct 8 into the first air guide duct 6.

With respect to the outflow region 4, a partition 10, which is preferably made of a hard material, in particular a hard plastic, like the vast majority of the other parts of the device as well, may be formed in the device 1.

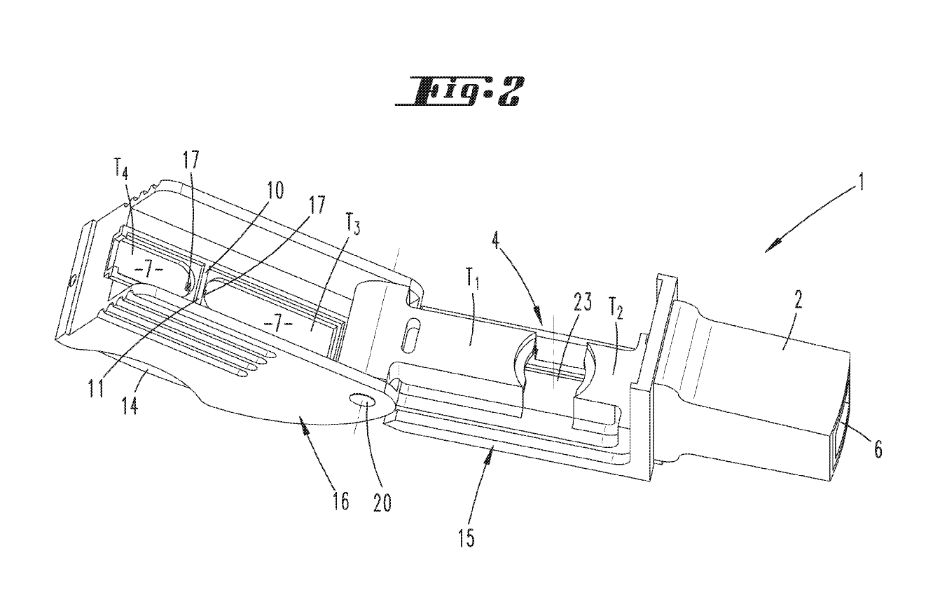

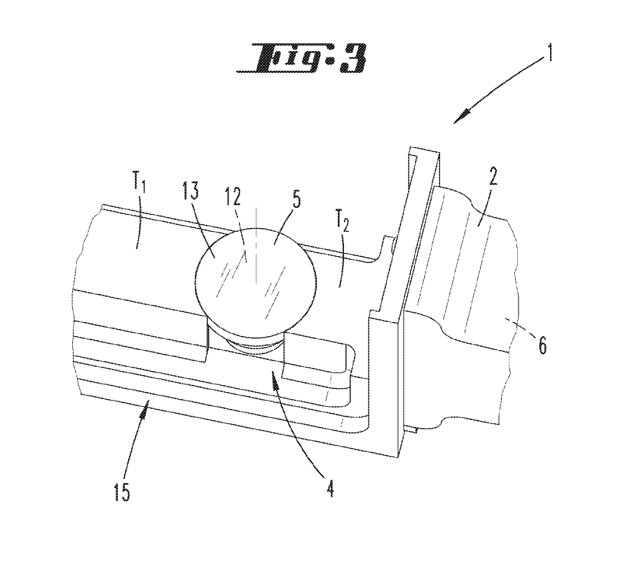

The partition 10 forms an end face 11, facing the chamber 5 (see also FIG. 2). The end face 11 may be provided with a soft material. The soft material may be, for example, rubber or a TPE.

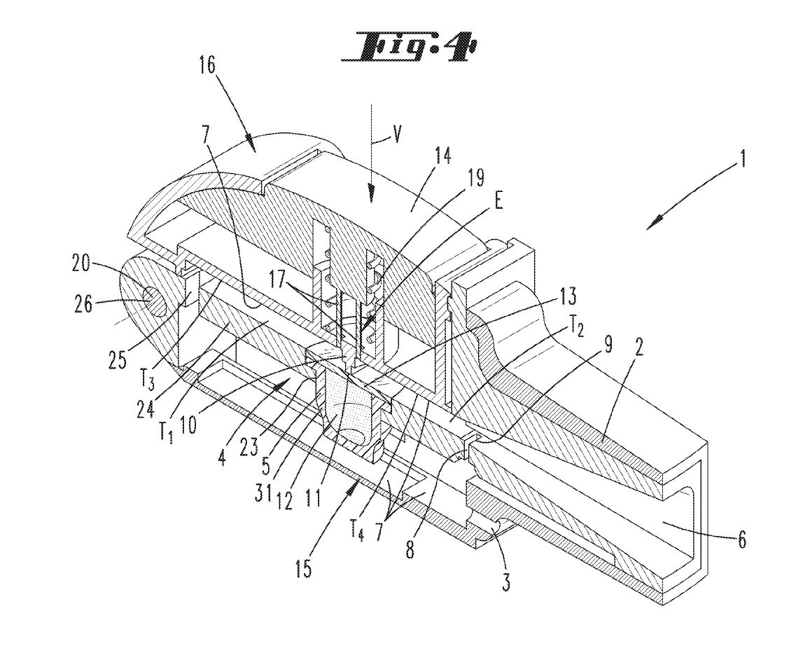

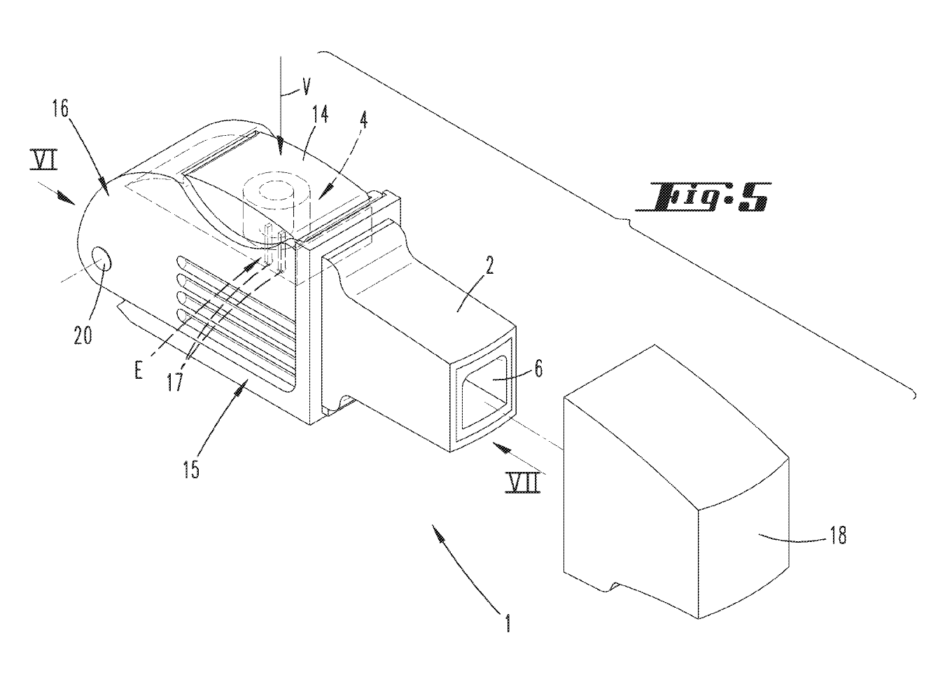

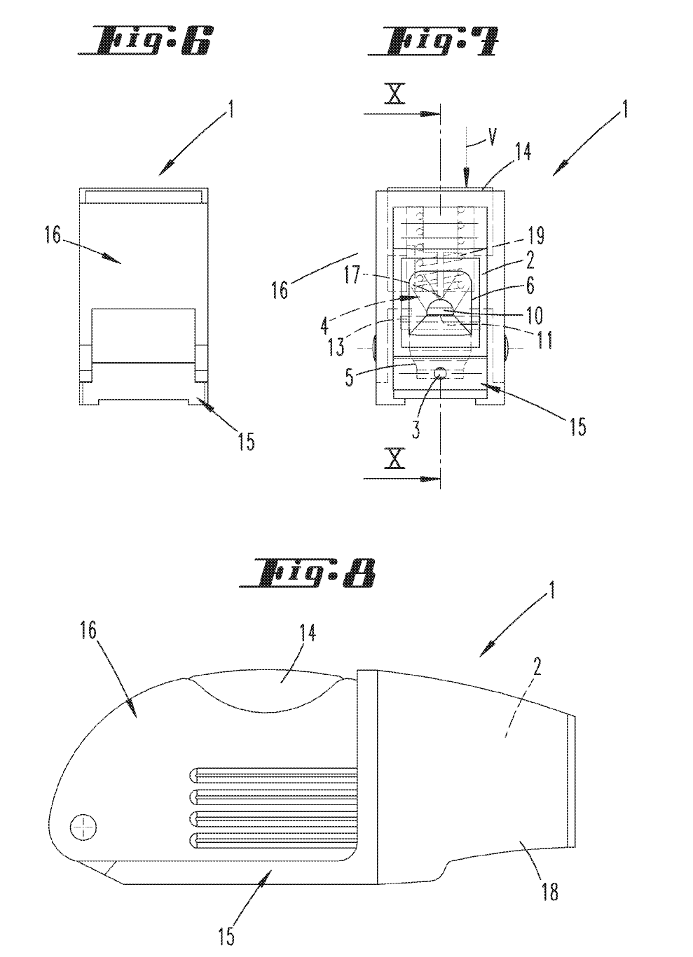

The end face 11 runs in a cover toward a chamber cavity 12, which, in the use condition, contains the substance to be discharged, as can also be seen in FIGS. 4 and 11.

The chamber cavity 12 has a cover 13, preferably in the form of a film cover. The cover 13 is more preferably openable, namely being puncturable in particular. In the region of the end face 11, which is in contact by means of the soft material, for example, the cover is preferably intact in the use condition.

The end face 11 of the partition 10 is in sealing contact with the cover 13 in the use condition, for example, according to FIG. 4, preferably also by means of the soft material. It traverses the cavity 12 of the chamber 5 in such a way that a projection of the partition 10 in the direction of a hold-down direction V of an actuating element 14, passes through the cavity 12 of the chamber 4.

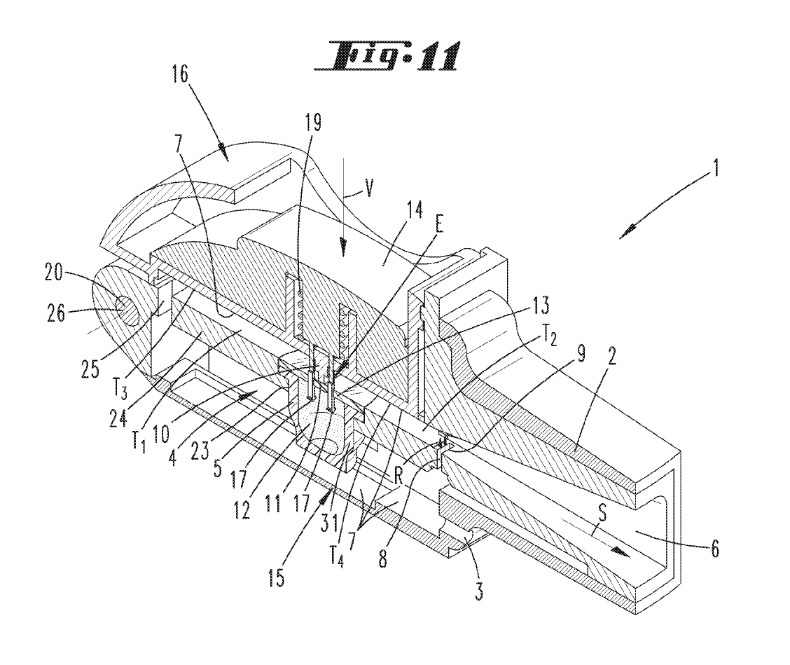

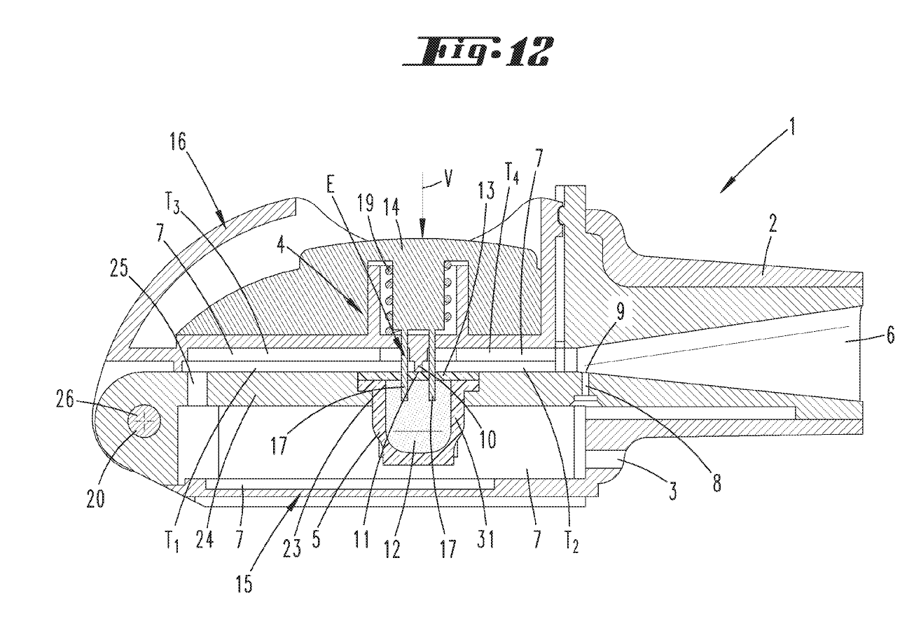

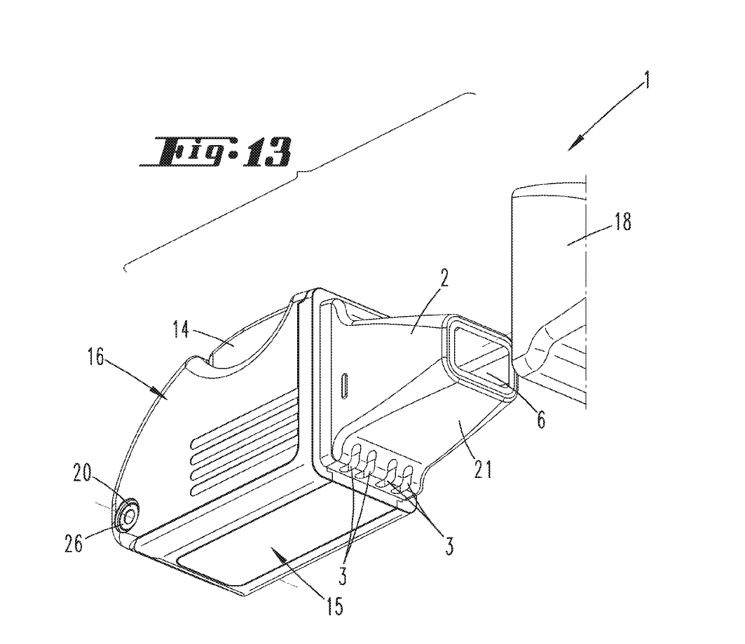

The first air guide duct 6 and/or the second air guide duct 7 is/are preferably designed, so that they are divided over their length, or a portion of their length at any rate. The result is a first subregion T1 and/or a second subregion T2 on the first housing part 15 (see FIG. 9, for example) and a third subregion T3 and/or a fourth subregion T4 formed on the second housing part 16 (see FIGS. 1 and 2, for example).

Whereas there may be two subregions, for example, subregions T1 and T2, as surface regions of a preferably flat surface, which obviously does not belong to an air guide duct but otherwise protrudes over the air guide duct. When seen separately, the additional subregions T3 and T4 may be designed in the form of chambers without any cover or bottom in the open condition. Due to the cooperation in the closed condition, the result is a closed first or second air guide duct 6, 7, respectively.

By pivoting the second housing part 16 into a position, preferably locked, but at any rate a contacting position, the aforementioned subregions T3 and T4 can be supplemented to yield the first housing part to the respective ready-to-use first and/or second air guide ducts 6, 7. At the same time, the partition 10 here can be movable relative to the chamber 5 into a contacting position and/or a release position. A bordering wall of the subregions T1, T2 and/or T3, T4 may also be provided with a sealing design, preferably made of a soft material.

A puncture device E for the chamber 5 for puncturing the cover 13 may have two puncture protrusions 17. The puncture protrusions 17 can be pressed into the puncture position against the spring force of a spring 19 that is supported on the device housing. After eliminating the load provided by a user's finger, for example, they return to their starting positions.

The puncture protrusions 17 are preferably each formed on one of the sides of the partition 10. Whereas an opening formed by a puncture protrusion 17 on one side of the partition 10 opening in the cover 13 of the chamber 5 is associated with the second air guide duct 7, the other opening is associated with the first air guide duct 6.

The puncture device E may be attached to the actuating element 14, as illustrated here, and can be depressed accordingly with the latter and/or reset by the spring force.

In the closed position of the device 1 with the chamber 5 inserted, except for the connecting duct 8, which acts as a short-circuit, an interruption in the main air flow path is achieved by means of the partition 10 sitting on the cover with a seal. This interruption is canceled only by forming the openings in the chamber cover 13. The chamber cavity 12 here acts as the channel section connecting the first and second air guide ducts.

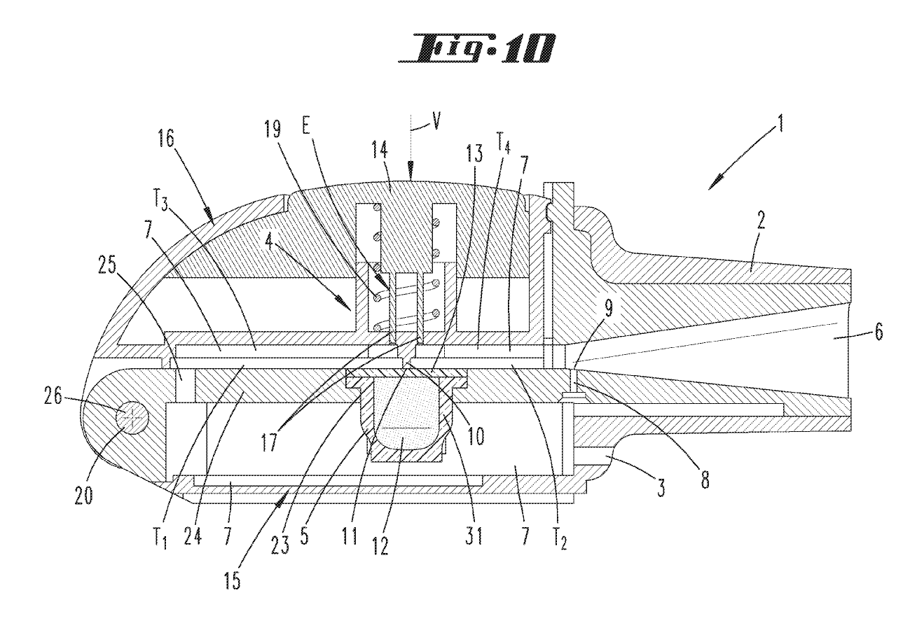

For use of the device 1, it is first opened according to FIG. 1. The chamber 5 is inserted into the outflow region 4, and then the device parts are guided toward one another by pivoting into a position according to FIG. 4 and/or FIG. 8. The user then creates the necessary openings in the cover 13 of the chamber 5 by depressing the puncture device E and then carries out an inhalation procedure by sucking on the discharge nozzle 2 after releasing said device. This draws air through the air inlet 3 and through the second air guide duct 7, so that the air is guided into two levels, one above the other, with a connecting region preferably being close to a hinge connection 20 of the housing parts 15 and 16, leading up to the outflow region 4. The air flow air guide duct 7 is preferably deflected by 180° in the connecting region, so that the direction of flow after the deflection is in the opposite direction from the air flow before the deflection.

In the outflow region 4, the air flows into the chamber 5 through a first opening in the cover 13 of the chamber 5, through the chamber cavity 12, and flows out at the other opening into the first air guide duct 6, and from there it goes as substance-loaded air to the discharge nozzle 2 and optionally into a user's mouth.

Before the substance-loaded air emerges, additional air flows out of the second air guide duct 7, through the connecting channel 8 and into the first air guide duct 6, namely air that has not passed through the outflow region 4. This results in a favorable turbulence and further breakup of the substance in the air, which is ultimately drawn in by the user.

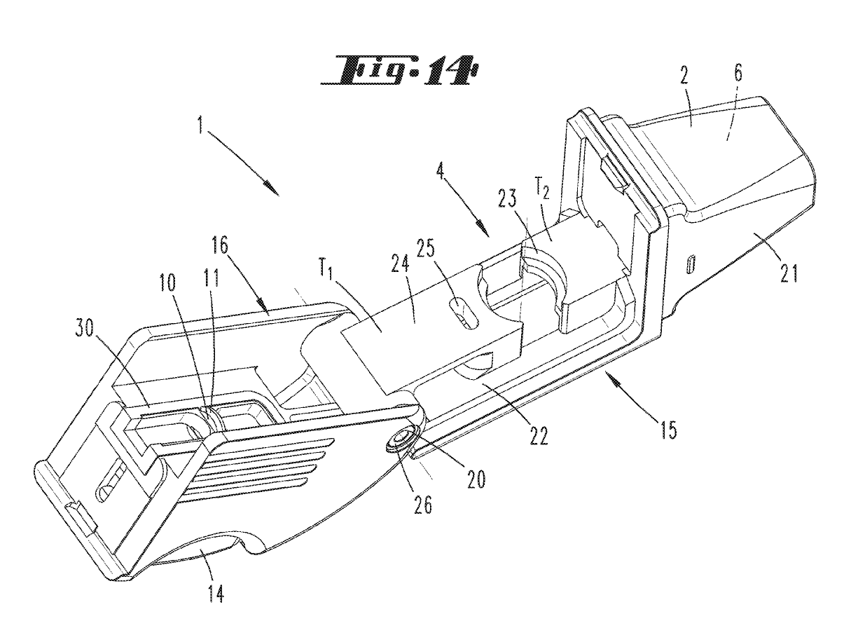

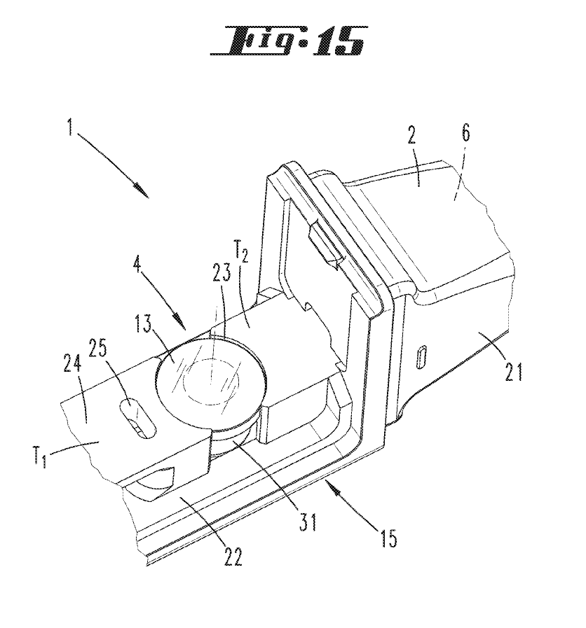

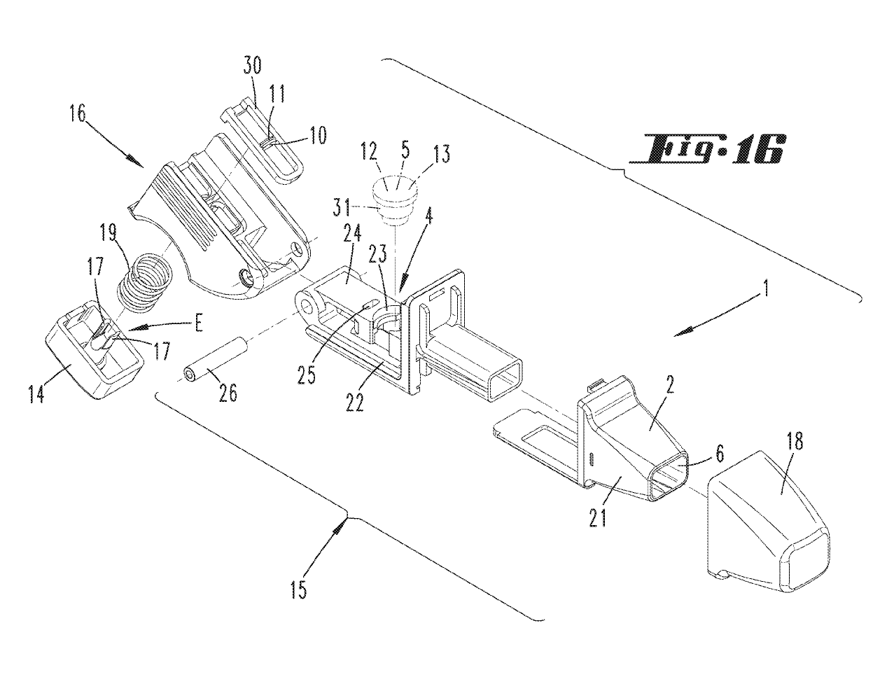

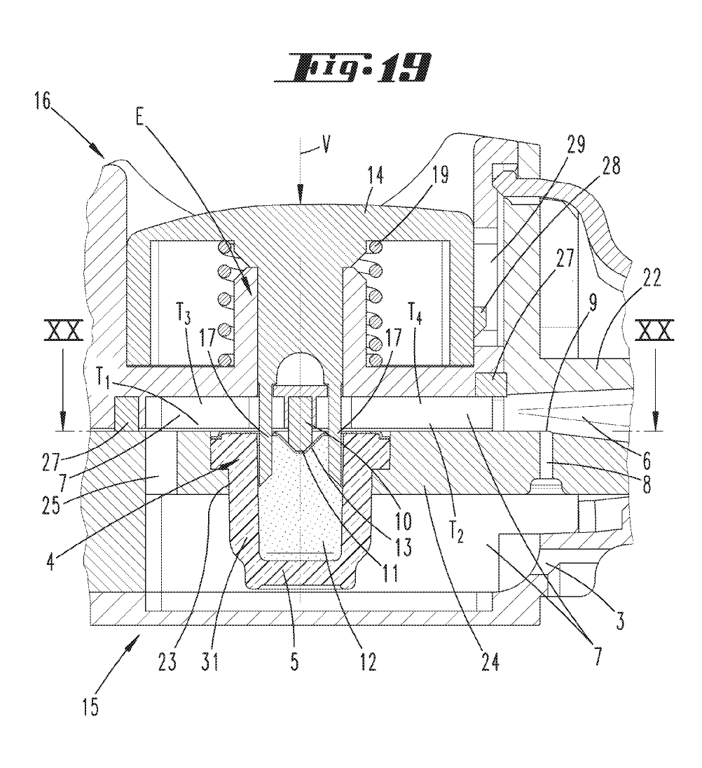

The diagrams in FIGS. 13 to 22 illustrate the device in a second embodiment.

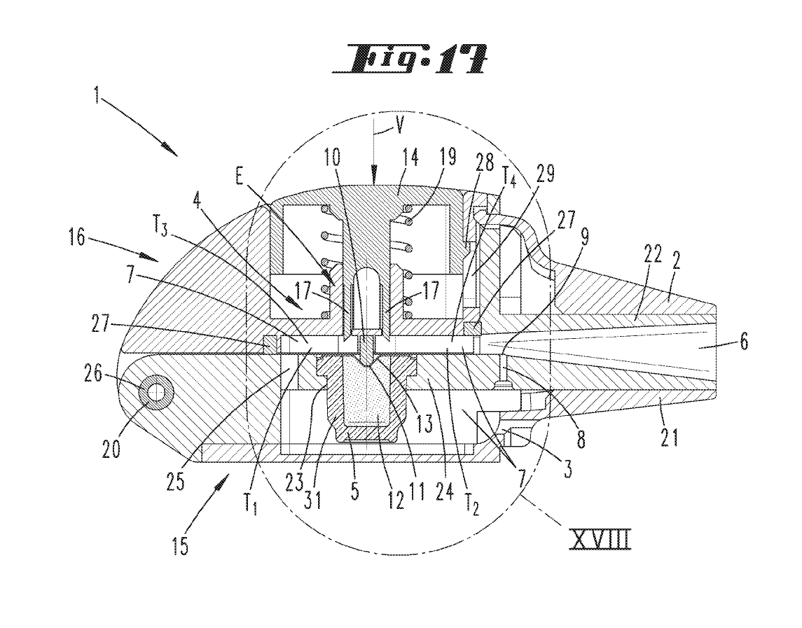

As can be seen in particular from the perspective exploded diagram in FIG. 16, the first housing part 15 is comprised essentially of a nozzle part 21 and an insert part 22 having the chamber receptacle 23. The chamber receptacle 23 in a use condition provides the air guide duct segments upstream and downstream from the insert partial cover 24, which divides the deflection. The insert partial cover has a through-opening 25 passing through it on the end of the air guide duct 7 formed in the use condition, facing the hinge region.

The insert part 22 may be secured by locking it into or onto the nozzle part 21.

In the embodiment illustrated here, four air inlets 3 arranged side-by-side across the direction of flow are designed in the nozzle partition at the bottom of the mouthpiece-type discharge nozzle 2.

The second housing part 16 is mounted pivotably by means of a journal 26 on the first housing part 15 to form the hinge connection 20.

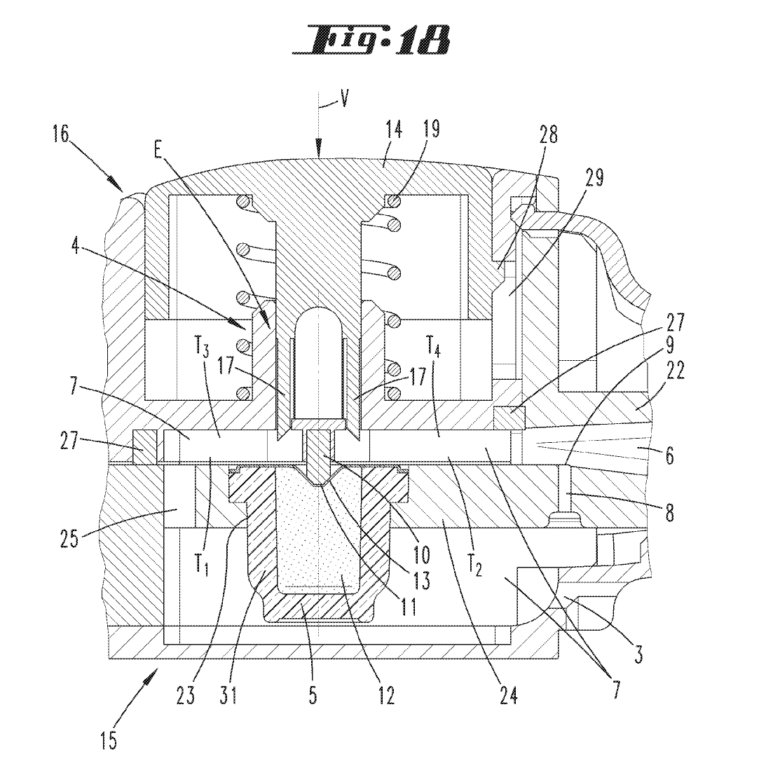

Gaskets 27 are provided in the area of the surfaces or surface segments, corresponding to the first housing part 15. These surfaces together with the first housing part 15 border the air guide duct 7 in particular in the direction of flow upstream and downstream from the chamber receptacle 23. These may be held on the second housing part 16, which can be pivoted away, for cooperating with corresponding surfaces on the first housing part 15 in the use position of the device 1 (cf. FIG. 17 in particular).

In the second embodiment as well, the actuating element 14 is held so that it is displaceable by sliding in the second housing part 16. A tab 28 provided on the outside of the wall on the actuating element 14 engages in a guide slot 29 of the second housing part 16, which extends in the hold-down direction for stop-limited displaceability in the relaxation direction of the spring 19 and thus for undetachable engagement, optionally also for rotationally secured holding of the actuating element 14 in the second housing part 16.

The partition 10 according to the second embodiment may be designed on a frame part 30, which can be inserted into the second housing part 16 and locked there, as illustrated in FIG. 16, for example.

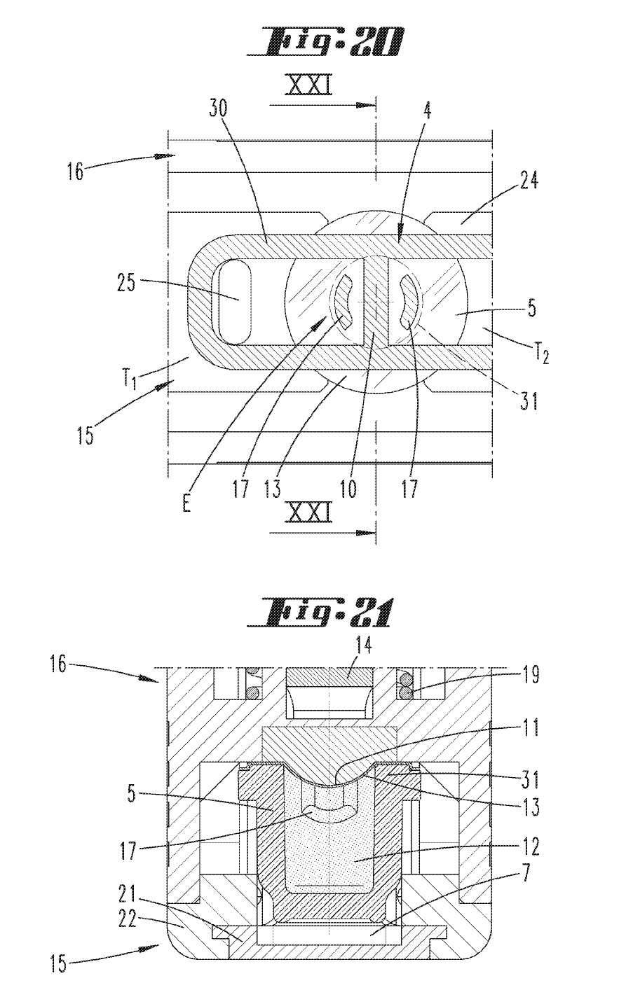

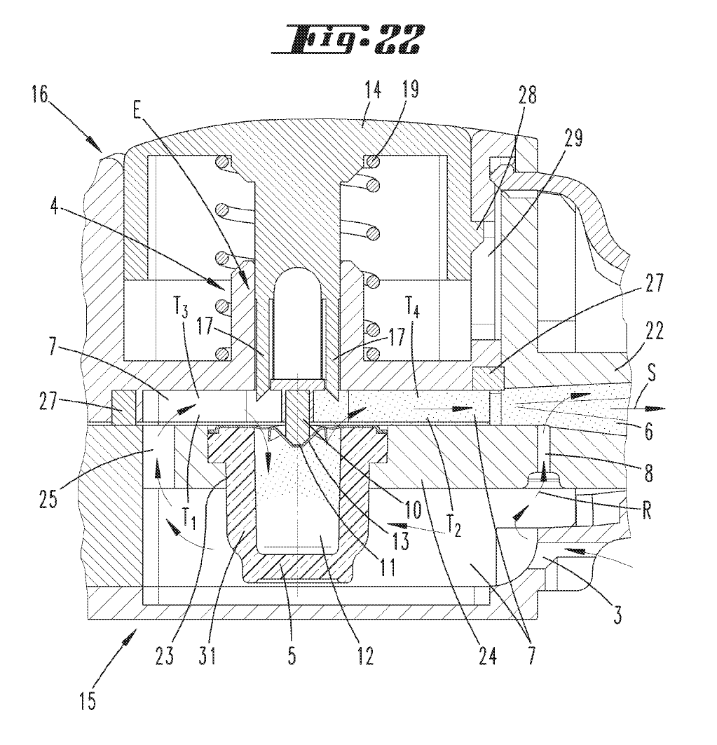

As shown in the sectional diagram in FIG. 21 in particular, the partition 10 is designed with a bulge in the hold-down direction, in particular a convex curvature, more specifically in the form of a segment of a circle, in the area of the end face 11, with respect to the direction of extent of the partition 10. This bulging region acts on the cover 13 of the inserted chamber 5 in such a way that the chamber forms a concave curvature toward the interior in the direction of the chamber cavity 12 when the device 1 is closed (cf. FIG. 21), i.e., when the second housing part 16 is pivoted inward. The concave curvature in the chamber cover 13 may be made possible as a result of a suitable elastic design of the cover 13, more specifically in the design of same as a film cover.

In one possible embodiment according to the diagram in FIG. 21, the partition 10 may be supported at the ends on the chamber wall 31 on both sides with respect to the cross section shown here, and is supported in the cross section, preferably over the full area, on the facing, convexly curved cover 13.

The inside contour of the chamber wall 31 may be selected to be circular, as illustrated in FIG. 20, to form a chamber cavity 12 in the shape of a circular cylinder.

In the second embodiment according to FIG. 20, the puncture protrusions 17 also extend on both sides of the partition 10 transversely to the hold-down direction V with respect to a cross section.

In this cross section, the puncture protrusions 17 have a contour adapted to the inside contour of the chamber wall 31, in particular having an outside contour adapted thereto. The cross section yields an outside contour in the form of a segment of a circular line. On the whole, the end of each puncture protrusion 17 may be designed like a pointed blade to facilitate a favorable cutting method for puncture of the chamber cover 13.

### LIST OF REFERENCE NUMERALS

1 device
2 discharge nozzle
3 air inlet
4 outflow region
5 chamber
6 air guide duct
7 air guide duct
8 connecting duct
9 inflow opening
10 partition
11 end face
12 chamber cavity
13 cover
14 actuating element
15 first housing part
16 second housing part
17 puncture protrusion
18 cap spring
19 hinge connection
20 nozzle part
21 insert part
22 chamber receptacle
23 insert part
24 cover
25 through-opening
26 journal
27 gasket
28 tab
29 guide slot
30 frame part
31 chamber wall
E puncture device
R inflow direction
S flow direction
T1 first subregion
T2 second subregion
T3 third subregion
14 fourth subregion
V hold-down direction

1-10. (canceled)

11: A device (1) for dispensing an air-dischargeable substance, having an air inlet (3), a discharge nozzle (2) through which substance-loaded air can be discharged, an outflow region (4), in which a chamber (5) containing substance to be discharged can be arranged, a first air guide duct (6) leading from the outflow region to the discharge nozzle, a second air guide duct (7) leading from the air inlet (3) to the outflow region (4), wherein the second air guide duct (7) is also fluidically connected by a connecting duct (8) to the first air guide duct (6), and with an inflow direction (R) of air flowing through an inflow opening (9) and through the connecting duct (8) into the first air guide duct (6), said air being directed perpendicular to the direction of flow (S) of the substance-loaded air into the first air guide duct (6) in the region of the inflow opening (9), wherein the inflow opening (9) is designed with a slot, and extends over more than 50% of a width of the first air flow duct (6), as seen across the direction of flow (S).

12: The device (1) for dispensing an air-dischargeable substance, having an air inlet (3), a discharge nozzle (2) through which substance-loaded air can be discharged, an outflow region (4), in which a chamber (5) containing substance to be discharged can be arranged in a chamber receptacle, a first air guide duct (6) leading from the outflow region (4) to the discharge nozzle (2), a second air guide duct (7) leading from the air inlet (3) to the outflow region (4), wherein a partition (10) is formed in the outflow region (4) between the first and second air guide ducts (6, 7), and the partition (10) is made of a hard material, wherein, furthermore, a free end face (11) of the partition (10) facing the chamber (5) is provided with a soft material, the chamber (5) has a film cover in a cover for a chamber cavity (12) containing substance, and the partition (10) is in sealing contact with the film cover by means of this soft material.

13: The device (1) for dispensing an air-dischargeable substance, having an air inlet (3), a discharge nozzle (2), through which substance-loaded air can be discharged, an outflow region (4), in which a chamber (5) with substance to be discharged can be arranged in a chamber receptacle, an air guide duct (6, 7) leading from the outflow region (4) to the discharge nozzle (2) and having a length, such that the chamber receptacle is designed in a first housing part (15), and a second housing part (16), which is pivotably connected to the first housing part (15), is provided, and wherein the air guide duct (6, 7) is designed with longitudinal divisions over a portion of its length, having subregions (T1, T2, T3, T4) formed on the first and second housing parts (15, 16), wherein a first air guide duct (6) leads further away from the outflow region (4) to the discharge nozzle (2); a second air guide duct (7) leads from the air inlet (3) to the outflow region (4); the first air guide duct (6) and the second air guide duct (7) are designed to be divided over at least a part of their length; and wherein a first subregion (T1) of the second air guide duct (7) and a second subregion (T2) of the first air guide duct (6) are provided on the first housing part (15); and a third subregion (T3) of the second air guide duct (7) and a fourth subregion (T4) of the first air guide duct (6) are provided on the second housing part (16); wherein the third subregion (T3) and the fourth subregion (T4) can further be supplemented by pivoting the second housing part (16) into a preferably locked position, but at any rate a contact position with the first housing part (15) to the respective first and/or second air duct (6, 7), wherein at the same time, a partition (10) which is impermeable for the air flow can be moved into a contact position with the chamber (5) inserted, relative to the chamber (5); a direct air flow from the second air guide duct (7) into the first air guide duct (6) is suppressed by the partition (10); and the air flow runs only through the chamber (5) when in use.

14: The device (1) for dispensing an air-dischargeable substance, having an air inlet (3), a discharge nozzle (2) through which substance-loaded air can be discharged, an outflow region (4) in which a chamber (5) with substance to be discharged can be arranged in a chamber receptacle, wherein the chamber (5) has a puncturable chamber cover (13), a puncture device (E) for forming a puncture opening in the chamber device, wherein a partition (10) is formed in the outflow region (4) for deflecting air into the chamber (5), and wherein the puncture device (E) additionally has two puncture protrusions (17), one of which is designed to be associated with one side of the partition (10) and the other of which is designed to be associated with the other side of the partition (10), wherein a puncture protrusion (17) is further adapted in cross section to an inside contour of a chamber wall (31) as seen in a hold-down direction (V), wherein the chamber wall (31) has a cross section in the form of a circular ring, and the puncture protrusion (17) has an outside contour in the form of a segment of a circle in a region puncturing the chamber cover (13) in displacement of the puncture protrusion (17) in a puncture direction; and the puncture protrusion (17) has a tip or a cutting edge which slides directly along an inside wall of the chamber (5) as part of the puncturing.

15: The device according to claim 12, wherein the partition (10) extends transversely to the direction of flow (S) in the air guide duct, with its free end face in a position in which it is in contact with the film cover.

16: The device according to claim 11, wherein a puncture device (E) is provided to form at least one puncture opening in a chamber cover (13).

17: The device according to claim 14, wherein the puncture device (E) is displaceable against a restoring force, for puncturing the film cover of the chamber (5) by means of the puncture protrusions (17).

18: The device according to claim 17, wherein the restoring force results from a spring (19).

19: The device according to claim 16, wherein the device (1) has a displaceable actuating element (14) for acting on the puncture device (E).

20: The device according to claim 17, wherein after the film cover has been punctured by the puncture protrusions (17), the chamber (5) connects the first air guide duct (6) to the second air guide duct (7).

21: The device according to claim 13, wherein the second housing part (16) is connected by a hinge connection (20) to the first housing part (15), and a deflection in the second air guide duct (7) is designed to be associated with the hinge connection (20).

22: The device according to claim 21, wherein the air flow in the direction of flow downstream from the deflection is preferably directed in the opposite direction from the air flow upstream from the deflection.

23: The device according to claim 16, wherein the cover (13) of the chamber (5) is designed to be run in a concave shape in a view from the outside.
